(19) United States
(12) Patent Application Publication
(10) Pub. No.: US 2007/0225669 A1
(43) Pub. Date: Sep. 27, 2007

(54) Heterogeneous absorbent cores

(75) Inventor: John Collins Dyer, Cincinnati, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company

(21) Appl. No.: 11/389,706

(22) Filed: Mar. 27, 2006

(51) Int. Cl. A61F 13/15

(52) U.S. Cl. 604/369; 604/370; 604/368; 604/365

(57) ABSTRACT

An absorbent core having an upper layer of open celled foam and a lower layer of network of microfibers.

### FIELD OF THE INVENTION

The present invention relates to heterogeneous absorbent cores, specifically an absorbent core having an upper layer of open-celled foam and a lower layer of a fibrous network comprising microfibers.

### BACKGROUND OF THE INVENTION

There is substantial commercial interest in developing absorbent cores for diapers, adult incontinence pads and briefs, surgical draperies and other medical items, and sanitary napkins. An important performance measure for these kinds of products is the ability of the products to acquire, distribute, and store fluids, such as those found in body exudates (e.g., urine, sweat, feces, blood, and menses). Presently, one technical approach for meeting these criteria includes using a combination of cellulosic fibers and interspersed superabsorbent particles. Superabsorbents are typically lightly crosslinked partially neutralized polyacrylic acid that forms a gel when exposed to free water.

Another approach is to employ open-celled polymeric foams and heterogeneous open-celled absorbent foams. Open-celled absorbent foams can be fabricated such that the foams have more than one distinct region. For absorbent articles including diapers, catamenial articles, and wound dressings, absorbent polymeric foams comprised of a single absorbent core material are known in the art. Open-celled absorbent foams can be used in a compressed or uncompressed state. “Superwicking” cross linked polyurethane foams are also known in the art for use in absorbent products.

One limitation to using open celled polymeric foams is that the foams can be expensive and/or difficult to manufacture. Furthermore, single layers of the cores can provide core integrity and flexibility, but may be unable to distribute and store absorbed fluid as effectively as desired.

Cores comprising a fibrous network of microfibers are known. Glass microfibers having an average diameter of less than about 0.75 microns are known to be suitable for fluid storage. Non-biopersistent glass microfibers can be used in absorbent articles to overcome challenges associated with microfibers that are not biosoluble. Interlaced mineral fibers having a specific surface area that is at least 0.25 m²/g and an average diameter less than 5 microns can also be used in absorbent cores. Another design for absorbent cores uses a composite core comprising webs of entangled blown microfibers and superabsorbent polymers. Another approach for forming an absorbent core is to use a web of entangled melt blown microfibers prepared from a nylon copolymer.

Multilayer absorbent structures are also known in the art. In a multilayered structure, one layer can provide for a surge layer for rapid fluid uptake. Surge layers characteristically have high permeability. Another layer can be formed to have sufficient capillary pressure to pull fluid in for storage.

There is a continuing unaddressed need for core structures that are capable of rapidly acquiring body fluids and are able to effectively store the fluid away from the wearer's body. Furthermore, there is a continuing unaddressed need for core structures having these properties which are economical to manufacture.

### SUMMARY OF THE INVENTION

An absorbent core comprising an upper layer of open celled foam and a lower layer of a fibrous network comprising a fibrous network of microfibers is disclosed. The lower layer can comprise vitreous microfibers. The microfibers can be non-biopersistent. The open celled foam can be formed of polyurethane. The open celled foam can be hydrophilic. The absorbent core can be an open celled foam having a mean cell diameter between about 250 microns and about 1,000 microns. The open celled foam can have a thickness between about 0.8 mm and about 3 mm. The microfibers can be formed into a wet laid web having a basis weight of between about 40 g/m² and about 350 g/m² and a density of between about 0.04 g/cc and about 0.25 g/cc. The lower layer can comprise at least about 5% superabsorbent polymer. The lower layer can comprise at least about 5% fibrous superabsorbent polymer. The lower layer can comprise at least about 10% fiber derived from the eucalyptus species of tree. The microfibers can be wet laid into a sheet. The lower layer can further comprise a binder. The lower layer can have a capillary pressure and the upper layer can have a capillary pressure and the lower layer capillary pressure can be at least about 2 times greater than the upper layer capillary pressure. An absorbent article can comprise the absorbent core. The absorbent article can be selected from the group consisting of an infant diaper, an adult diaper, a wound dressing, an incontinence product, and a menstrual pad. The absorbent article can comprise a backsheet and a topsheet. The absorbent core can comprise a lower layer comprising a superabsorbent polymer and non-biopersistent inorganic vitreous microfibers and an upper layer comprising an open celled hydrophilic polyurethane foam. The open celled hydrophilic polyurethane foam can have a density of between about 40 kg/m³ and about 100 kg/m³. The inorganic vitreous microfibers can have an average effective diameter between about 0.1 micron and about 6 microns. The absorbent core can be employed in an absorbent article comprising an outer core, an inner core, and an over wrap, where the outer core and the inner core are disposed in a coaxial arrangement with the outer core circumferentially surrounding the inner core, wherein the over wrap circumferentially surrounds the outer core, wherein the inner core is comprised of a fibrous network comprising microfibers and the outer core is comprised of an open celled foam.

### BRIEF DESCRIPTION OF THE DRAWINGS

FIG. 1 of the drawings shows an infant diaper comprising the absorbent core.

FIG. 2 of the drawings shows a menstrual pad comprising the absorbent core.

FIG. 3 of the drawings shows a tampon comprising the absorbent core.

### DETAILED DESCRIPTION OF THE INVENTION

As used herein, absorbent articles include infant diapers, incontinent briefs, incontinent pads, training pants, diaper inserts, panti-liners, catamenial pads, sanitary napkins, menstrual pads, tampons, bandages, facial tissues, surgical dressings and drapes, paper towels, and the like.

As used herein, the terms “body liquids,” “body fluids,” or “body exudates” include, but are not limited to, urine, menses, vaginal discharges, blood, sweat, mucous, wound exudates, feces, and combinations of these substances.

As used herein, the term “absorbent core” refers to the component of the absorbent article that is primarily responsible for the liquid handling properties of the article, including acquiring, distributing, and storing body liquids. As such, the absorbent core typically does not include the topsheet or backsheet of the absorbent article.

As used herein, the term “superabsorbent” refers to a material capable of absorbing at least ten times its dry weight of a 0.9% saline solution at 25° C. Superabsorbent polymers absorb fluid via an osmotic mechanism to form a gel. Superabsorbents may be particulates, fibers, foams, sheets, or other shapes.

As used herein, the term “microfibers” refers to fibers having an average effective diameter of about 0.1 micron to about 6 microns and an aspect ratio of at least about 100. The aspect ratio of a microfiber is the contour length of the fiber divided by the average effective diameter of the fiber. The contour length of a fiber is the length of the fiber in a substantially straightened condition. Long microfibers can have aspect ratios which exceed 1×10¹². The microfiber may be of any configuration, including but not limited to straight, curled, kinked, crimped, and combinations thereof. The cross sectional area of the microfiber orthogonal to its contour length at any point may have any geometric shape, including but not limited to circular (round), square, flat, oval, star-shaped, irregular, and combinations thereof. For fibers having a non-circular cross section, the effective diameter is the diameter of a circle having a cross sectional area equal to that of the fiber. Microfibers may comprise any material, including but not limited to natural polymers, synthetic polymers, minerals, glass, ceramics, metals, vegetable matter, animal matter, carbon, and combinations thereof. A sample of microfibers having an average effective diameter between about 0.1 and about 6 microns may contain individual fibers with diameters greater than about 6 microns and/or individual fibers with diameters less than about 0.1 micron.

As used herein, the term “inorganic” refers to a material which is not organic in nature. As used herein, the term “organic” refers to compounds of carbon.

As used herein, the term “vitreous” refers to a material which is substantially non-crystalline in that the material comprises more than about 90% amorphous material. A vitreous material can comprise more than about 99% amorphous material.

“Non-biopersistent” refers to microfibers comprising at least 18% alkaline and alkaline earth oxides and meet at least one of the criteria for lack of biopersistence listed below. A non-biopersistent material according to the present invention can also meet the criteria of the German Dangerous Substances Ordinance (Gefahrstoffverordnung) Annex V, No. 7.1(1). A suitable method for selecting a fiber composition to test for non-biopersistence of certain fibers according to the test method below is to use the method reported by Eastes, W., Potter, R. M., and Hadley, J. G. (2000), “Estimation of Dissolution Rate from In-Vivo Studies of Synthetic Vitreous Fibers,” Inhalation Toxicology, 12(11), 1037-1054. An online calculator implementing the method can be found at http://fiberscience.owenscorning.com/kdisapp.html. This calculator predicts the rate of biodissolution as a function of the chemical composition of the fiber. A non-biopersistent fiber meets at least one of the following criteria: (1) a short-term biopersistence test by inhalation showing that the fibers longer than 20 μm have a weighted half-life of less than 10 days (a suitable short-term biopersistence test by inhalation is described in European Union protocol ECB/TM/26 rev. 7), or (2) a short-term biopersistence test by intratracheal instillation showing that the fibers longer than 20 μm have a weighted half-life less than 40 days (a suitable short-term biopersistence test by intratracheal instillation is described in European Union protocol ECB/TM/27 rev. 7), or (3) an appropriate intra-peritoneal test showing no evidence of excess carcinogenicity (a suitable test for carcinogenicity of inorganic vitreous microfibers after intra peritoneal injection in rats is described in European Union protocol ECB/TM/18(97)), or (4) a suitable long-term inhalation test showing the absence of relevant pathogenicity or neoplastic changes (A suitable long-term inhalation test is described in European Union protocol ECB/TM/17(97)). These test methods are reported in European Commission Joint Research Centre Institute for Health and Consumer Protection Unit: Toxicology and Chemical Substances, European Chemicals Bureau (1999), “Methods for the Determination of the Hazardous Properties for Human Health of Man Made Mineral Fibres (MMMF),” Report 18748, David M. Bernstein and Juan M. Riego Sintes Eds.

As used herein, the term “foam” is synonymous with the term “cellular polymer,” which includes materials having a significant void volume, typically greater than about 75%. “Open-celled” foams further have a reticulated internal structure disposed therein comprising relatively thin “strut” elements interconnected and forming cells or pores providing for fluid communication throughout the structure. Mean cell diameters refer to the diameter of the pores in the foam visible by microscopy. The pores tend to be relatively spherical in shape and the mean diameter can be measured by using microscopic techniques. One suitable technique is to use a scanning electron micrograph and measure the apparent mean diameter of at least about 25 representative cells to determine the mean. The density of foams can be determined using uncompressed samples of said foams devoid of contaminants such as water, and measuring the volume and weight of the foam. A cubic sample having an edge length greater than or equal to about 2 cm is practical.

As used herein, “hydrophilic” refers to a material or substance having affinity for water or aqueous fluids. In general, a hydrophilic surface will have a contact angle with water of less than about 60°, or even less than about 30°.

As used herein, the term “layer” refers to a three-dimensional structure having two dimensions that are substantially greater than the third dimension. The term layer is not limited to single layers or sheets of material. Thus a layer may comprise laminates or combinations of several sheets or webs of the requisite type of materials. Accordingly, the term “layer” includes the terms “layers” and “layered.” A “layer” can include a three-dimensional shape which is not completely planar. That is, the “layer” need not be completely rectilinear and can encompass more complex shapes, while still being substantially larger in two dimensions than in the third dimension.

As used herein, the term “upper layer” refers to the layer which is in closer proximity to the body of the wearer of the article in use than the lower layer, such that the upper layer receives body fluids (urine, menstrual fluid, blood, fecal matter, wound exudates, and the like) before the lower layer. The upper layer can be referred to as a fluid acquisition layer. The “lower layer” refers to the layer that serves to pull the body fluids away from the upper layer to distribute and/or store the fluid. A portion of the lower layer remains in close capillary contact with the upper layer. The lower layer can be referred to as a fluid storage layer. For an absorbent article in which the layers of the article are arranged in a coaxial relationship, such as a tampon, the upper layer is analogous to an outer core and the lower layer is analogous to an inner core.

As used herein, the term “flexible” refers to materials which are compliant and readily conform to the general shape and contours of the wearer's body under normal body-imposed forces.

As used herein the term “joined” refers to the condition where a first member is attached, or connected, to a second member either directly or indirectly. Where the first member is attached, or connected, to an intermediate member which in turn is attached, or connected, to the second member, the first member and second member are joined indirectly.

The absorbent core 20 can comprise a lower layer 40 of a fibrous network comprising microfibers. One suitable microfiber is a non-biopersistent inorganic vitreous microfiber having an average effective diameter of between about 0.1 microns and about 6 microns. Inorganic vitreous microfibers particularly useful in the present invention comprise about 18% or more of alkaline oxides and alkaline earth oxides and are non-biopersistent as described previously. Vitreous microfibers can exhibit good fluid handling properties, resiliency, softness, and lack of biopersistence. Inorganic compositions suitable for making microfibers useful in the present invention are described in European Pat. 1048625B, U.S. Pat. No. 6,261,335, and published U.S. Pat. Application No. 2003/0015003. Suitable non-biopersistent inorganic vitreous microfibers are available from Lauscha Fiber International, Summerville, S.C.

Known web forming techniques, including wet laying which can be suitable for staple fibers, may be used to form a fibrous network comprising vitreous microfiber staple feedstock. The steps of wet laying comprise dispersing vitreous microfiber staple in an aqueous medium. The dispersion is then laid down on a forming screen from a head box or other suitable distribution means. Then the aqueous medium drains through the forming screen to form a nascent web, which is then dried and wound to form a rolled web of vitreous microfibers.

In an alternative embodiment, a binder is added to the web to improve mechanical stability of the web. The binder can be thermoplastic binder fibers or powder added to the furnish when the vitreous microfibers are wet laid. The drying step can then be used to melt the binder thereby stabilizing the web. The binder can comprise a hydrophilic material. The binder can comprise only a single thermoplastic material. The binder may comprise a bicomponent fiber comprising two thermoplastic materials in which one of the materials has a melting point substantially higher than that of the other material so as to preserve fiber integrity when the fiber is exposed to a temperature that causes flow of the thermoplastic material having a lower melting point.

The binder can be a latex binder applied to the wet nascent web as a component of the furnish or after web formation. The latex binder can then be cured in a drying step. The binder can be a polymeric solution (e.g., aqueous polyvinyl alcohol) sprayed on the web which is dried along with the microfibers in the drying step. The binder can be a binding adhesive, such as a hot melt material sprayed on the dried web before the dried web is wound.

The binder can be a thermosetting wet strength resin applied as a component of the furnish or after formation of the web. For example, a spray application of KYMENE 557H, available from Hercules, Wilmington, Del., can increase the strength of a fibrous network comprising microfibers. If a small amount of cellulosic fibers such as fibers from the Eucalyptus species of tree or crill or similarly classified material is included, additional wet strength materials known in the papermaking arts may be included as a component of the furnish or after web formation. Nonlimiting examples of such materials include and PAREZ 631 NC available from Lanxess, Pittsburgh, Pa., and the aforementioned KYMENE 557H.

Because binders may affect the fluid handling properties of the web, only the minimum amount necessary to obtain sufficient mechanical strength should be used. For a thermoplastic binder, the level can be between about 0.1% and about 20%. Alternatively, the level for the thermoplastic binder can be between about 0.1% and about 15%. For post formation binders, the add-on to the dry web can be between about 0.1% and about 20% or even between about 0.1% and about 15%.

In addition to webs having binder fibers, other types of fibers may be incorporated into the web to enhance the wet strength and the final strength of the web. Suitable high surface area fibers include microfibrous cellulose, high surface area cellulosic fibers (e.g., conventional cellulosic pulp fibers, particularly eucalyptus fibers, crosslinked cellulosic fibers, including those crosslinked with polyacids such as citric or polyacrylic), and highly refined cellulosic fibers having Canadian Standard Freeness between about 1 and about 200 can be useful. Fibers having Canadian Standard Freeness of between about 40 to about 100, referred to herein as “crill,” can also be useful.

A portion of the fibers in the web may also be comprised of synthetic polymeric or semi-synthetic polymeric fibers. For example, synthetic fibers such as polyester, polypropylene, and polyethylene may be used in relatively small amounts to provide additional strength to the structure. Semi-synthetic fibers, such as rayon, are also suitable. One suitable synthetic fibrous material is CREATE-WL, a short staple (about 3 mm to about 18 mm) polypropylene fiber having a length suitable for wet laying marketed by FiberVisions, Covington, Ga. Other types of synthetic fibers include those termed “bicomponent” fibers wherein a portion of the fiber is of one type and another portion is of another type, often in a coaxial arrangement. An example of a bicomponent fiber is a fiber comprising a core of polyethylene and a sheath of polypropylene surrounding the core. Other synthetic fibers which may be included are nylon, polyolefins, polyacrylonitrile, polyesters, polyamide, polyaramid, polyacrylates including both polyalkylacrylates and sodium polyacrylates, superabsorbent fibers, and the like. The quantity of such fibers depends on the desired final properties of the web. Alternatively, the fibers can be used in an amount be between about 1% and about 25%. In another embodiment, the fibers can be used at a level between about 1% and about 15%. The fibers can be used at a level between about 1% and about 10%.

The web may be airlaid directly after formation of the microfibers by collecting the fibers on a suitable forming device or by using conventional airlaying techniques used for staple-length fibers. In either case, the web may be formed by a collection of fibers on a foraminous structure. A vacuum system underlying the foraminous structure can aid in gathering the fibers into a web form. Airlaid webs of this type can also use binders and other fibrous materials as described above for wet laid webs.

The web can have a basis weight of between about 40 g/m² and about 350 g/m². Alternatively, the web can have a basis weight between about 80 g/m² and about 160 g/m². Layers of the web may be stacked to achieve higher overall basis weights. The density of the web can be between about 0.04 g/cc and about 0.25 g/cc. Alternatively, the density of the web can be between about 0.07 g/cc and about 0.10 g/cc. Microfibers can comprise at least about 10% of the fibrous assembly.

The absorbent core 20 can comprise an upper layer 30 comprising open celled foam. The upper layer 30 can be an open celled hydrophilic polyurethane foam. The open celled foam can be hydrophilic and comprised of polyvinyl alcohol-formaldehyde or melamine/formaldehyde composition. The mean cell diameters for open celled foams can be between about 250 and about 1,000 microns. The mean densities of open celled foams can be between about 40 kg/m³ and about 100 kg/m³. The open celled foam can be white or near white and photo stable with respect to yellowing. The foam can be sliced or otherwise formed into sheets between about 0.8 mm and about 3 mm in thickness and formed into a substantially planar shape having dimensions dictated by the particular product application. The sheet will generally be planar, but can have a contoured shape such as a hump in the middle for better fit in some applications. The open celled foams can also be ground and reformed into a sheet using a suitable adhesive or collected in a permeable bag or other container for use. Exemplary foams include Hydrasorb (Hypol) from W. R. Grace, Columbia, Md., EPI-LOCK from Calgon/Vestal Laboratories, St. Louis, Mo., LYOFOAM from ConvaTec, Skillman, N.J., and POLYCRIL 400 from Fulflex, Lincoln, R.I. Foams containing superaborbents are available from Woodbridge Foam, Mississauga, Ontario, Canada. Other useful foams include those disclosed by Gustafsson and Widlund in WO9623466A1, Malmgren in US20020143310A1, Bhagwati and Gehrke in U.S. Pat. No. 6,027,795A, Bhagwati and Gehrke in U.S. Pat. No. 5,573,994A, and Hissink et al. in US20060008419A1. The nature of the optimum open celled foam properties can depend on the particular product in which the foam is to be employed.

The ability of a structure to “pull” fluid against an opposing force, such as gravity or against affinity for fluid of another substrate with which the structure is in intimate capillary contact, can be characterized by the capillary pressure. The capillary pressure can be characterized as the hydrostatic head at which the vertically wicked fluid loading is 50% of the free absorbent capacity under equilibrium conditions at 31° C. The hydrostatic head is represented by a column of fluid (e.g., synthetic urine). The upper layer 30 can have a capillary pressure of about 2 cm to about 5 cm and lower layer 40 can have a capillary pressure of about 6 cm to about 15 cm, or higher. A higher ratio can be beneficial. The benefit of a higher ratio may be offset by the consequence that higher ratios can also result in long times for the materials to reach equilibrium.

In one embodiment, the capillary pressure exerted by the lower layer 40 of the absorbent core 20 exceeds that of the upper layer 30 of the absorbent core 20 by a factor of at least about two. Alternatively, the capillary pressure exerted by the lower layer 40 of the absorbent core 20 can exceed that of the upper layer 30 of the absorbent core 20 by a factor of at least about 3, so as to assure that most of the fluid that initially resides in the upper layer 30 of the absorbent core 20 is transported to the lower layer 40 of the absorbent core 20 for distribution and storage.

Structures having relatively high surface areas per unit volume, relatively low contact angles with the fluid of interest, and absorbing fluids having relatively high surface tensions will concomitantly have relatively high capillary pressures. High pressures are of little use in the lower layer of the absorbent core unless the structure is maintained in close capillary contact with the upper layer 30 of the absorbent core 20 such that significant gaps of air between them are not formed. Air gaps between the upper layer 30 and lower layer 40 can break the capillary connection and impair fluid transfer between the layers.

The open celled foam disclosed herein can be used in an initially compressed state that expands to full volume as a function of wear time and/or fluid loading. The foam may collapse after an insult of fluid as the lower layer pulls fluid away from the foam.

Structures of representative absorbent articles such as infant diapers are described in more detail in U.S. Pat. No. 5,387,207 (Dyer et al.). An illustration of a infant diaper is shown in FIG. 1. The diaper 100 is comprised of a topsheet 10, an absorbent core 20, and a backsheet 50. The absorbent core 20 is disposed between the topsheet 10 and backsheet 50. An absorbent core 20 can be formed by attaching the upper layer 30 and lower layer 40. The upper layer 30 and lower layer 40 can be attached by using an adhesive spray at the contact points, or around the perimeter, or by spot patterning, or by any other technique sufficient to maintain substantial capillary connectivity between the upper layer 30 and lower layer 40 and to prevent the layers from shifting in plane relative to one another during storage or use.

Adhesive tabs 60 can be joined to the diaper to supply structure for fitting the diaper 100 to an infant or an adult. Alternatively, adhesive tabs 60 can be replaced with hook and loop type fastening components, interlocking fasteners such as “tabs and slots”, buckles, buttons, snaps, and/or cohesive fastening components, which are not illustrated. An optional component, which is not shown, is a fluid acquisition layer commonly referred to as a “secondary topsheet.” A secondary topsheet can be between the absorbent core 20 and topsheet 10. The backsheet 50 and the topsheet 10 are positioned adjacent the garment facing surface and the body facing surface, respectively, of the absorbent article and can be joined to one another using an adhesive. The diaper 100 can be formed in a manner to fit the wearer using adhesive tabs 60. The body facing surface of an absorbent article is oriented towards the wearer's body when the article is worn. The garment facing surface of an absorbent article is the surface of the absorbent article when worn that is oriented away from the wearer's body. Among the differences between an infant diaper, a menstrual pad, and a bandage are the size of the absorbent article and the means for attaching the absorbent article so that the article remains in contact with the wearer.

The backsheet 50 can be impervious to fluids and can be manufactured from a thin plastic film. Other flexible liquid impervious materials may also be used. The backsheet 50 can comprise woven or nonwoven materials, polymeric films such as thermoplastic films of polyethylene or polypropylene, or composite materials such as a film-coated nonwoven material. The backsheet 50 can be a polyethylene film having a thickness between about 0.012 mm (0.5 mil) and about 0.051 mm (2.0 mils). The backsheet 50 may be embossed and/or matte finished to provide a more cloth like appearance. The backsheet 50 can permit vapors to escape from the absorbent core 20 (i.e., the backsheet is breathable) and still prevent exudates from passing through the backsheet 50. Suitable materials for use as backsheet 50 are available form Clopay Plastic Products Company of Mason, Ohio.

The topsheet 10 can be compliant, soft feeling, and non-irritating to the wearer's skin. Further, the topsheet 10 is fluid pervious, permitting fluids (e.g., menses and urine) to readily penetrate through the thickness of topsheet 10. A suitable topsheet 10 can be manufactured from a wide range of materials such as woven and nonwoven materials, polymeric materials such as apertured formed thermoplastic films, apertured plastic films, hydroformed thermoplastic films, porous foams, reticulated foams, reticulated thermoplastic films, and thermoplastic scrims. Suitable woven and nonwoven materials can be comprised of natural fibers (e.g., wood or cotton fibers), synthetic fibers (e.g., polymeric fibers such as polyester, polypropylene, or polyethylene fibers) or a combination of natural and synthetic fibers. The backsheet 50 and topsheet 10 can be joined to one another by an adhesive, thermal bonding, ultrasonic bonding, or by any other suitable method known in the art. The topsheet 10 can have a basis weight from about 10 to about 25 g/m², a minimum dry tensile strength of at least about 150 g/cm in the machine direction and a strikethrough of less than about 3 seconds according to European Disposables and Nonwovens Association standard method 150.4-99. One suitable topsheet 10 comprises a polypropylene spunbonded nonwoven comprising fibers of less than 3 denier having a basis weight of about 18 g/m², as is available from BBA Fiberweb of Simpsonville, S.C.

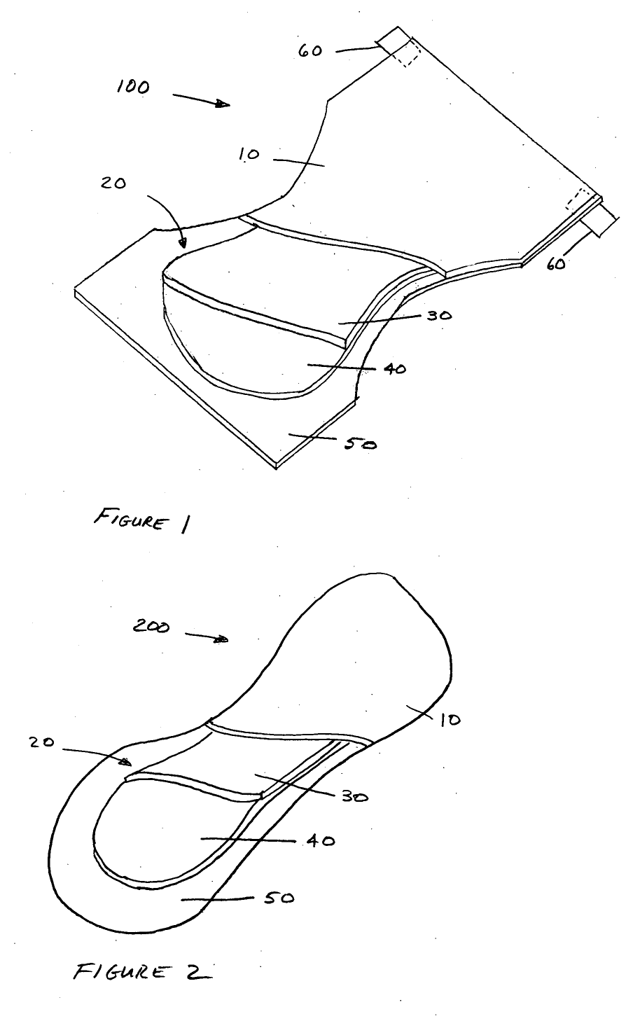

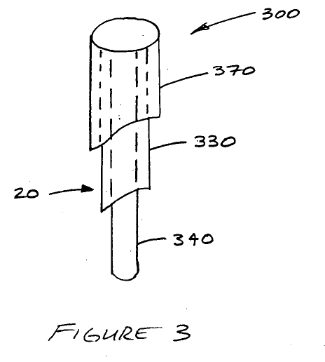

An illustration of a menstrual pad 200 comprising the absorbent core 20 is shown in FIG. 2. The structure of the menstrual pad 200 is similar to that of the diaper 100 shown in FIG. 1, but differs in geometry. The menstrual pad 200 comprises a topsheet 10 and a backsheet 50 and an absorbent core 20 disposed there between. The upper layer 30 and lower layer 40 can be attached by using an adhesive spray at the contact points, or around the perimeter, or by spot patterning, or by any other technique sufficient to maintain substantial capillary connectivity between the upper layer 30 and lower layer 40 and to prevent the layers from shifting in plane relative to one another during storage or use. The absorbent core 20 comprises an upper layer 30 and a lower layer 40. The menstrual pad 200 can have an adhesive, which is not shown in FIG. 2, applied to the garment facing surface of the backsheet 50 to affix the menstrual pad 200 to the wearer's panty. A hotmelt adhesive such as HL-1491 XZP available from H. B. Fuller, Toronto, Canada can be suitable. The adhesive can be placed on the backsheet 50 in a pattern of stripes, dots, lines, about the periphery, or any other pattern by which the menstrual pad can be attached to the wearer's undergarment.

An illustration of a tampon 300 comprising the absorbent core 20 is shown in FIG. 3. For a tampon, the outer foam core 330 and inner microfiber core 340 are disposed coaxially with the outer foam core 330 circumferentially surrounding the inner microfiber core 340. An over wrap 370 can circumferentially surround the outer foam core 330. The over wrap 370 can be a material such as rayon, cotton, bicomponent fibers, polyethylene, polypropylene, other suitable natural or synthetic fibers known in the art, and mixtures thereof. The over wrap 370 can be comprised of bicomponent fibers that have a polypropylene core surrounded by polyethylene manufactured by Vliesstoffwerke Christian Heinrich Sandler GmbH & Co.KG (Schwarzenbach/Saale, Germany) under the trade name SAS B31812000. The over wrap 370 can comprise a nonwoven over wrap of a hydroentangled blend of 50% rayon, 50% polyester available as BBA 140027 produced by BBA Corporation, Simpsonville, S.C. The over wrap may be 100% polyester. The over wrap may be treated to be hydrophilic, hydrophobic, wicking, or non-wicking.

In use, the diaper 100 and menstrual pad 200 can be held in place by any support or attachment device known for such purposes, including a pressure sensitive adhesive attached to the backsheet or adhesive tabs.

The diaper 100, menstrual pad 200, and tampon 300 as well as any other absorbent article disclosed herein can be assembled by hand or by using an automated process.

### EXAMPLE 1

A microfiber-containing nonwoven forming the lower layer 40 is attached to an open-celled hydrophilic polyurethane foam forming the upper layer 30 wherein the foam is sliced to a thickness of 1.2 mm and cut to the shape desired for the absorbent product to be formed. The attachment can be made by using a light adhesive spray, such as polyvinyl acetate, where the upper layer 30 is in contact with the lower layer 40.

Urine added to the upper layer is absorbed rapidly and then more slowly substantially transferred to the lower layer as a result of capillary pressure (in addition to any movement by the wearer or the product which can facilitate such fluid movement by a pumping action).

Blood added to the upper layer is also absorbed rapidly and then transferred to the lower layer. The transfer of blood from the upper layer 30 to the lower layer 40 tends to mask the appearance of the blood to a viewer who examines the top of the absorbent product (in this case a menstrual pad, tampon, bandage, wound dressing, surgical drapery, or the like).

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. An absorbent core comprising an upper layer and a lower layer, said upper layer comprising an open celled foam and said lower layer comprising a fibrous network comprising microfibers.

2. The absorbent core of claim 1 wherein said lower layer comprises vitreous microfibers.

3. The absorbent core of claim 1 wherein said microfibers are non-biopersistent.

4. The absorbent core of claim 1 wherein said open celled foam is comprised of polyurethane.

5. The absorbent core of claim 1 wherein said open celled foam is comprised of a material selected from the group consisting of polyvinyl alcohol-formaldehyde and melamine/formaldehyde compositions.

6. The absorbent core of claim 1 wherein said open celled foam is hydrophilic.

7. The absorbent core of claim 1 wherein said open celled foam has a mean cell diameter between about 250 microns and about 1,000 microns.

8. The absorbent core of claim 1 wherein said open celled foam has a thickness between about 0.8 mm and about 3 mm.

9. The absorbent core of claim 1 wherein said microfibers are formed into a wet laid web having a dry basis weight of between about 40 g/m² and about 350 g/m² and a dry density of between about 0.04 g/cc and about 0.25 g/cc.

10. The absorbent core of claim 1 wherein said lower layer comprises at least about 5% superabsorbent polymer.

11. The absorbent core of claim 1 wherein said lower layer comprises at least about 10% fiber derived from the Eucalyptus species of tree.

12. The absorbent core of claim 1 wherein said microfibers are wet laid into a sheet.

13. The absorbent core of claim 1 wherein said lower layer further comprises a binder.

14. The absorbent core of claim 1 wherein said lower layer has a capillary pressure and said upper layer has a capillary pressure, wherein said lower layer capillary pressure is at least about 2 times greater than said upper layer capillary pressure.

15. An absorbent article comprising a topsheet, a backsheet, and an absorbent core disposed between said topsheet and said backsheet, said absorbent core comprising an upper layer comprising an open celled foam and a lower layer comprising a fibrous network comprising microfibers.

16. The absorbent article of claim 15 wherein said absorbent article is selected from the group consisting of infant diapers, adult diapers, wound dressings, panti-liners, incontinence products, and a menstrual pads.

17. An absorbent core comprising:
a) a lower layer comprising a superabsorbent polymer and non-biopersistent inorganic vitreous microfibers; and
b) an upper layer comprising an open celled hydrophilic polyurethane foam.

18. The absorbent core according to claim 17 wherein said open celled hydrophilic polyurethane foam has a density of between about 40 kg/m³ and about 100 kg/m³.

19. The absorbent core according to claim 17 wherein said inorganic vitreous microfibers have an average effective diameter between about 0.1 micron and about 6 microns.

20. An absorbent article comprising an outer core, an inner core, and an over wrap, wherein said outer core and said inner core are disposed in a coaxial arrangement with said outer core circumferentially surrounding said inner core, wherein said over wrap circumferentially surrounds said outer core, wherein said inner core is comprised of a fibrous network comprising microfibers and said outer core is comprised of an open celled foam.
